(19) United States
(12) Patent Application Publication
(10) Pub. No.: US 2013/0218000 A1
(43) Pub. Date: Aug. 22, 2013

(54) MRI Coil For Imaging Of The Breast And Thoracic Area

(71) Applicants: Daniel D. COPPENS, Avondale, PA (US); David R. RABENO, Avondale, PA (US)

(72) Inventors: Daniel D. COPPENS, Avondale, PA (US); David R. RABENO, Avondale, PA (US)

(73) Assignee: QFIX SYSTEMS, LLC, Avondale, PA (US)

(21) Appl. No.: 13/771,056

(22) Filed: Feb. 19, 2013

Related U.S. Application Data
(60) Provisional application No. 61/599,946, filed on Feb. 17, 2012.

(51) Int. Cl. A61N 5/10; A61B 5/055; A61B 6/03; A61B 5/00

(52) U.S. Cl. CPC A61N 5/1039; A61B 5/4836; A61B 5/0555; A61B 5/0037; A61B 6/037; A61B 6/032; A61B 5/0035; A61B 5/4312
USPC 600/411

(57) ABSTRACT

The present invention provides a novel MRI coil configuration for breast cancer imaging in preparation for radiation therapy planning and treatment. Both an adjustable anterior and posterior coil sets are described. The coil sets are supported and configured so as not to affect the accurate and repeatable positioning of the patient and breasts. The coil sets are removable so that they can be extracted before radiation therapy treatment commences.

### CROSS REFERENCE TO RELATED APPLICATION

The present application claims priority from U.S. Provisional Application Ser. No. 61/599,946, filed on 17 Feb. 2012.

### FIELD OF THE INVENTION

The present invention relates to magnetic resonance imaging (MRI) systems and, in particular, to the radio-frequency (RF) coils used in such systems.

### BACKGROUND OF THE INVENTION

Magnetic Resonance Imaging (MRI) provides one of the best imaging technologies for distinguishing soft tissue. This is extremely important for diagnosis and treatment of breast cancer. Since the breast is comprised of soft tissue, it is difficult to distinguish various masses using diagnostic x-ray equipment. Traditional mammography equipment uses very low energy x-rays but these are not available on CT scanners and Radiation Therapy (RT) simulators.

MRI has not traditionally been used for RT simulation since the spatial accuracy of MRI has not been high enough to use it for planning and aiming the treatment beam. However, new MRI machines from manufacturers such as GE, Philips and Siemens have made significant advances in spatial accuracy, opening the way for RT simulation through MRI. This in turn drives the need for Radiation Therapy positioning devices that are MRI compatible and incorporate innovative MRI coils that allow MRI imaging with the patient in position on the treatment devices that will be employed during treatment.

The most common position for Breast Cancer treatment by RT is supine on a device commonly called a supine breast board. Supine breast boards typically contain an angling patient surface that acts somewhat like the back of a lawn chair. The patient's back is placed at an angle so that the breast drops in a repeatable manner from simulation to treatment and during each treatment fraction. In order to accomplish MRI simulation with a supine breast board, it is desirable to have an MRI coil to enhance the imaging of the breast.

### SUMMARY OF THE INVENTION

The present invention solves the aforementioned problems and provides a device for supporting and imaging a patient in an MRI machine in preparation for radiation therapy treatment

The MRI coil configuration described herein can be used for breast imaging and treatment performed with an angling breast board. The present invention accommodates a wide variety of patient sizes and anatomy as well as being able to work with the angling feature of the breast board. Since the patient's anatomy during treatment must be in the same position during simulation imaging, it is preferable that the MRI coils do not touch the patient.

Generally, it is preferred to perform MRI imaging with both an anterior and posterior coil set to enhance the quality of the images. For flat surface devices (for example, Stereotactic Body Radiation Therapy (SBRT) positioning devices), the coil that is built into the table of the MRI machine can often be used. However, the quality of the image is greatly enhanced by being able to get the coils as close to the patients' anatomy as possible. Therefore, it is advantageous to mount a coil to either the top or bottom surface or both the top and bottom surfaces of the patient support surface. Coils used can either be rigid coils, flexible coils or a combination of the two. In the case of a breast board with angling backrest, it can be critical to mount a coil on the posterior side of the patient. In a preferred embodiment of the angling breast treatment device, the posterior coil is mounted to the underside of the angling section of the device by various attachment similar to the anterior coil.

The coil placed above the patient can be attached to a rigid attachment, a telescoping cylinder, a rotatable member, or an articulating coil support mechanism. This attachment mechanism is releasably attached to the device (platform) on either side of the patient's head or neck superior to the shoulder. The coil support mechanism may be attached either to the left side of the patient, the right side, or both sides. By attaching to either the left or right side of the patient a coil may be brought as close as possible to the breast of interest. The coil support mechanism can also be integrated into the coil set itself. In a preferred embodiment, the attachment comprises a quick disconnect. This can be accomplished by a variety of methods well known methods. The coil support structure is then cantilevered over the patient's thoracic region. Since it is not attached at the sides of the patient's thorax, a large variety of patient widths and girths can be accommodated without the coil or support mechanism touching the patient. In the case of the angling breast device, the coil set can be placed anterior to the patient. Another advantage of this configuration for breast imaging is that the coil set can be ideally placed for imaging of the supraclavicular region which contains the lymph nodes. It is often important during breast cancer treatment to treat the lymph nodes.

Specifically, the present invention provides a device for supporting and imaging a patient in an MRI machine in preparation for radiation therapy treatment comprising; a platform with a top and bottom surface for supporting the patient; at least one removable anterior coil set support mechanism attached to the platform; and at least one anterior MRI coil attached to the coil set support mechanism and suspended above or below the patient; wherein the MRI coil does not contact the patient, and wherein the MRI coil support attaches to the platform around the patient head or neck on at least one side of the patient head or neck.

### BRIEF DESCRIPTION OF THE DRAWINGS

FIG. 1 is an isometric view of a patient positioned for supine breast treatment and imaging. An MRI Coil is attached to the positioning device and is positioned above the patient's breasts.

FIG. 2 is an isometric view of a patient positioned for supine breast treatment and imaging. An MRI Coil is attached to the positioning device and is positioned above the patient's left breast.

FIG. 3 is a side view of a patient positioned for supine breast treatment and imaging with an MRI Coil positioned above her left breast. A posterior coil is mounted below the patient support platform.

FIG. 4 is a top view of a patient positioned for supine breast treatment and imaging demonstrating an electrical connection to the MRI Coil. The coil is positioned above the patient's breasts.

FIG. 5 is a top view showing a patient positioned for supine breast treatment and imaging demonstrating an electrical connection to the MRI Coil. The coil is positioned above the patient's breasts using two supports one on either side of the patient's head.

FIG. 6 is a top view of a patient positioned for supine breast treatment and imaging demonstrating an electrical connection to the MRI Coil. The coil is positioned above the patient's left breast.

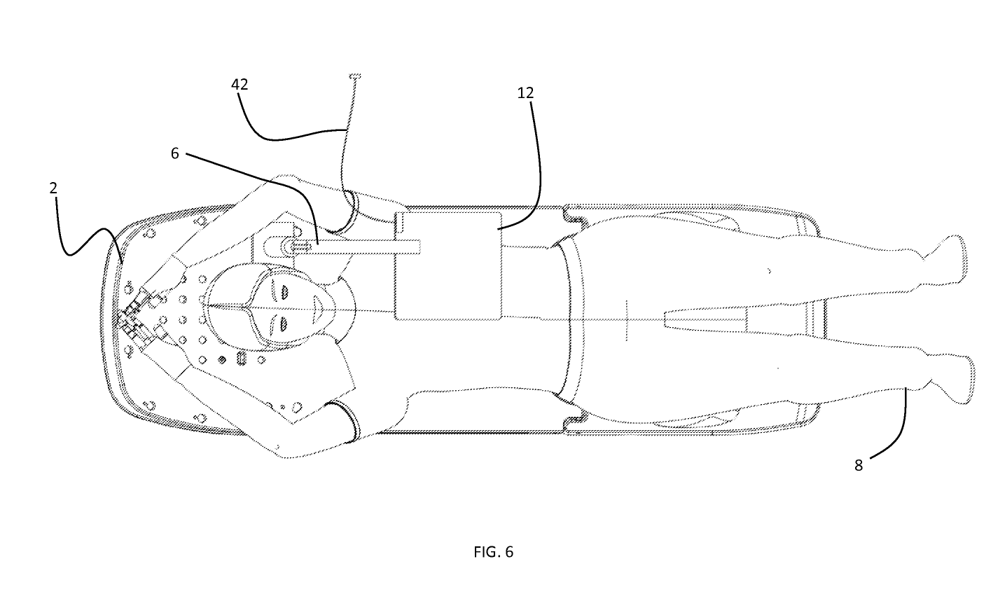

FIG. 7 is an isometric view showing a flexible MRI coil positioned above the patient's breasts.

FIG. 8 is an isometric view of an MRI Coil attached to a patient support platform designed for positioning patients for Stereotactic Body Radiation Therapy.

FIG. 9 is a side view of MRI Coils attached to a patient support platform designed for positioning patients for Stereotactic Body Radiation Therapy. The anterior coil is attached with a rigid arm and a posterior coil attached to the underside of the patient support platform.

FIG. 10 is an isometric view showing the MRI Coil attached to the patient support platform using a rigid arm.

FIG. 11 is an isometric view showing the MRI Coil attached to the patient support platform using a telescoping arm.

FIG. 12 is an isometric view showing the MRI Coil attached to the patient support platform using an articulating arm.

FIG. 13 shows a variation of the anterior MRI coil (130) in which the support mechanism is integrated into the coil itself. This can be a rigid or articulating configuration. In this figure, the coil takes the form of a bib.

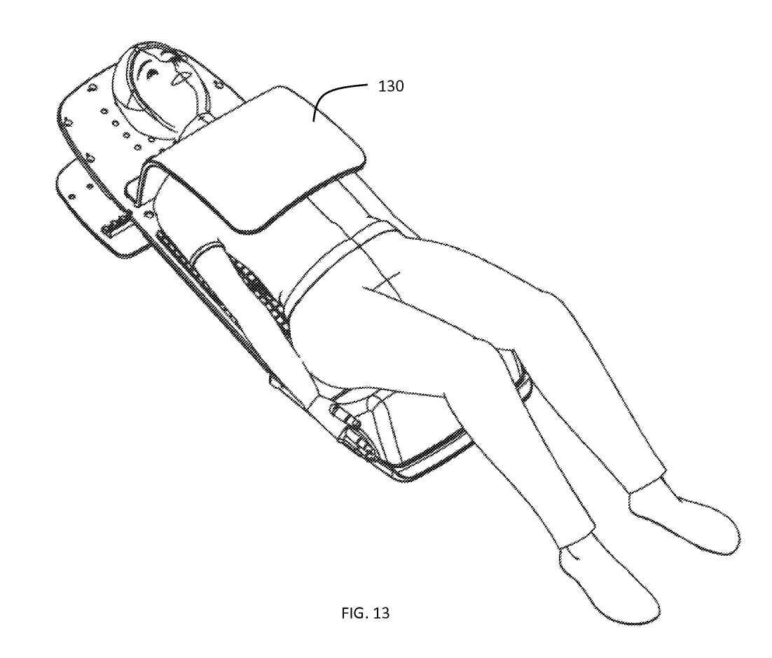

FIG. 14 shows the anterior coil with posterior coil.

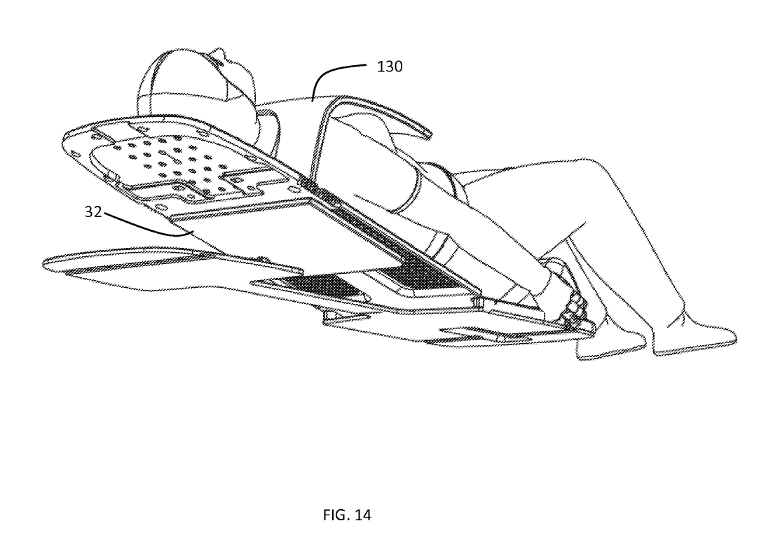

### DETAILED DESCRIPTION OF THE INVENTION

The present invention provides_a platform with a top and bottom surface for supporting the patient; at least one removable anterior coil set support mechanism attached to the platform; and at least one anterior MRI coil attached to the coil set support mechanism and suspended above or below the patient. It is critical to the present invention that the MRI coil does not contact the patient. This is accomplished with the MRI coil support attaching to the platform around the patient head or neck on at least one side of the patient head or neck. In this configuration, the MRI coil does not attach around the torso and therefore, the torso does not obstruct the imaging and treatment field laterally. Furthermore, by attaching the coil around the patient's head or neck you do not limit the size of the patient that can be accommodated.

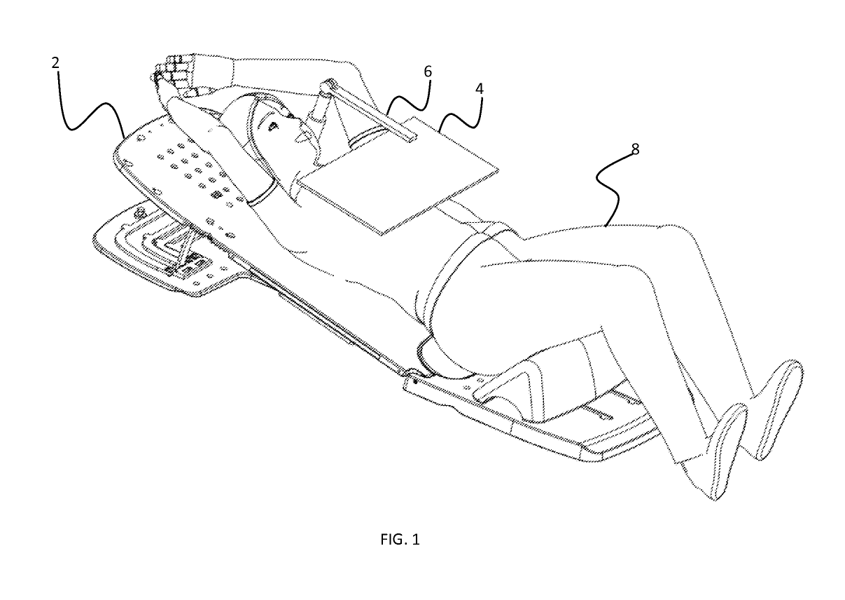

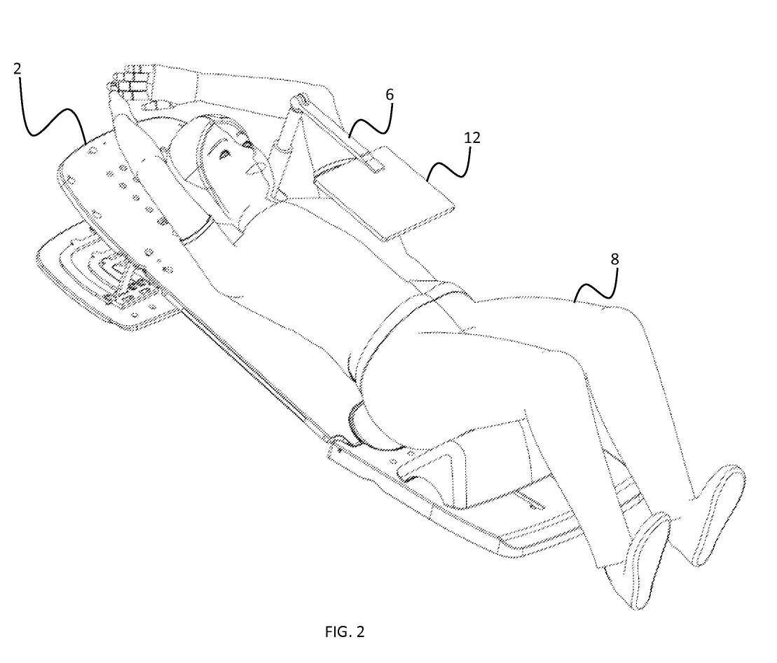

A preferred embodiment of the invention is shown in FIG. 1. A patient 8 is positioned on a patient support platform 2 in preparation for imaging in a MR imaging device. An MRI coil 4 is positioned above the patient's breasts by a support mechanism 6. In this image the coil extends across the patient's chest. In FIG. 2 it is shown that a one-breast coil 12 can be placed such that it only covers the patient's breast that is to be treated.

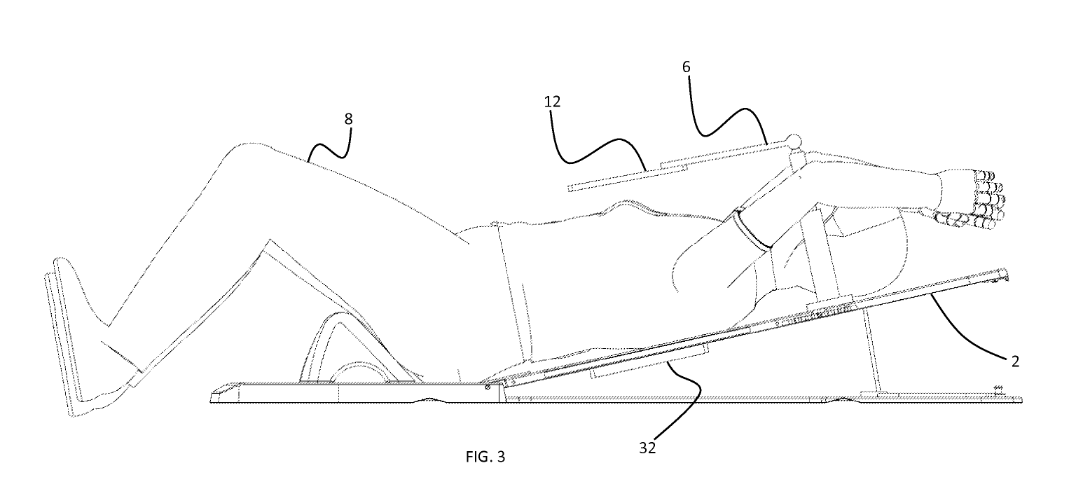

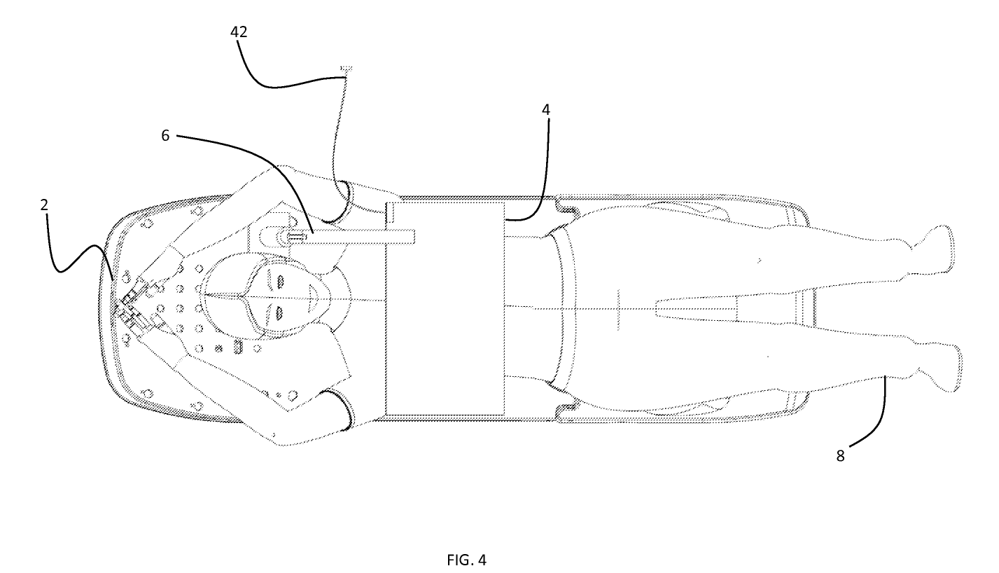

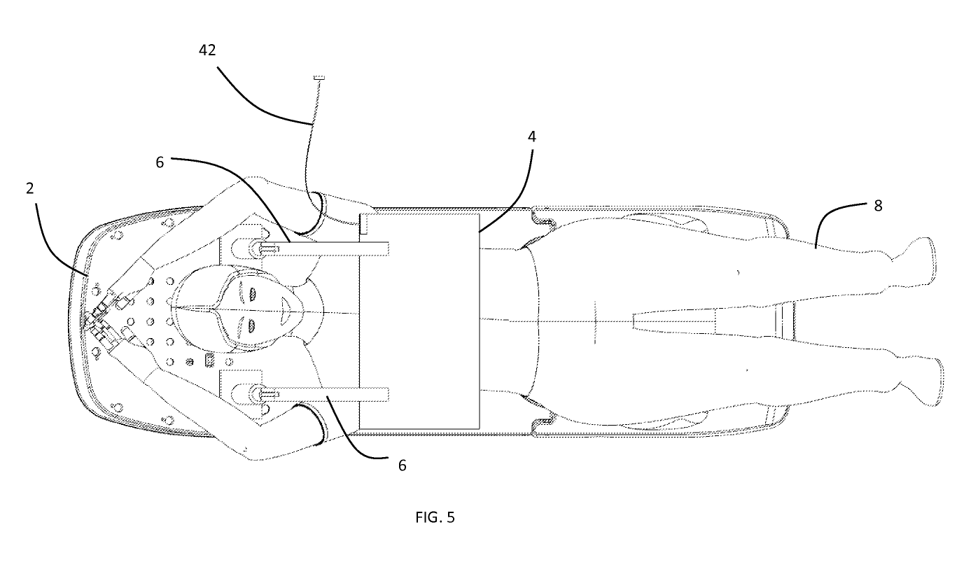

In FIG. 3 it is shown that in addition to the anterior coil 12 a posterior coil 32 can be removably attached to the patient support platform 2. In FIG. 4 an electrical connection 42 is connected to the MRI coil which can interface with the MRI machine. As shown in FIG. 5 the anterior coil can be positioned by two supports 6 one on either side of the patient.

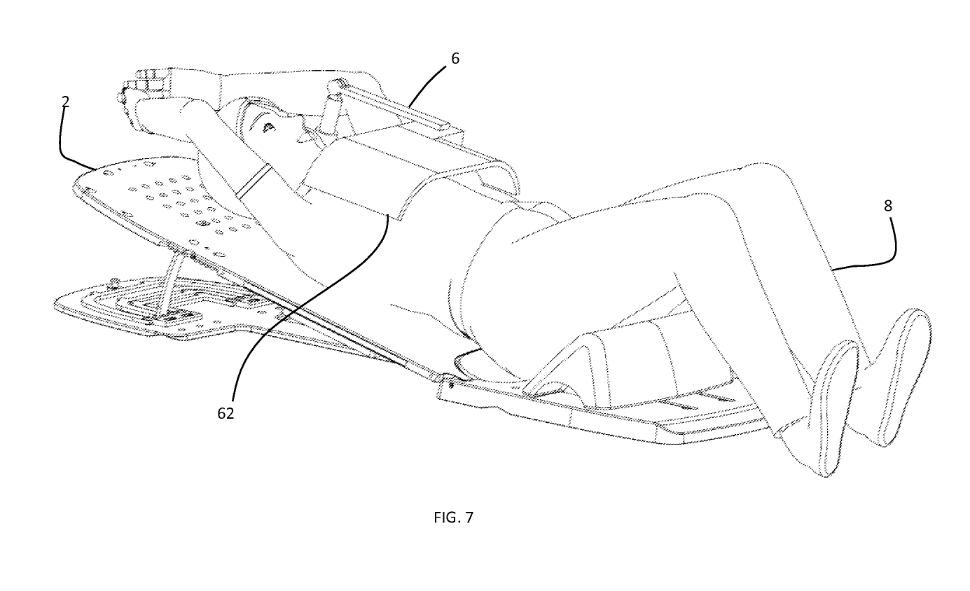

In another preferred embodiment, shown in FIG. 7, the support 6 is used to position a flexible coil 62 above the patient's chest. This allows for a more conformed coil to the patient's anatomy and can increase the quality of the images.

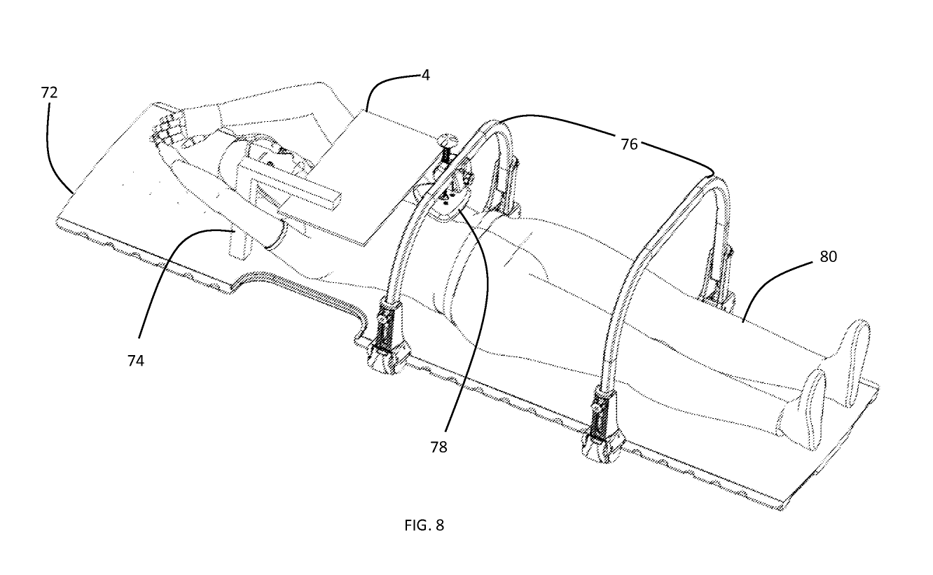

In yet another preferred embodiment, shown in FIG. 8, involves attaching a support 74 to a patient support platform 72 which is used for Stereotactic Body Radiation Therapy. In addition, support bridges 76 can be attached to the platform. A compression paddle 78 can be attached to the bridges 76. This paddle allows the diaphragm to be compressed during treatment which reduces tumor motion and contributes to more accurate treatment delivery. In this configuration the device can be used for imaging of the lung, liver, and pancreas.

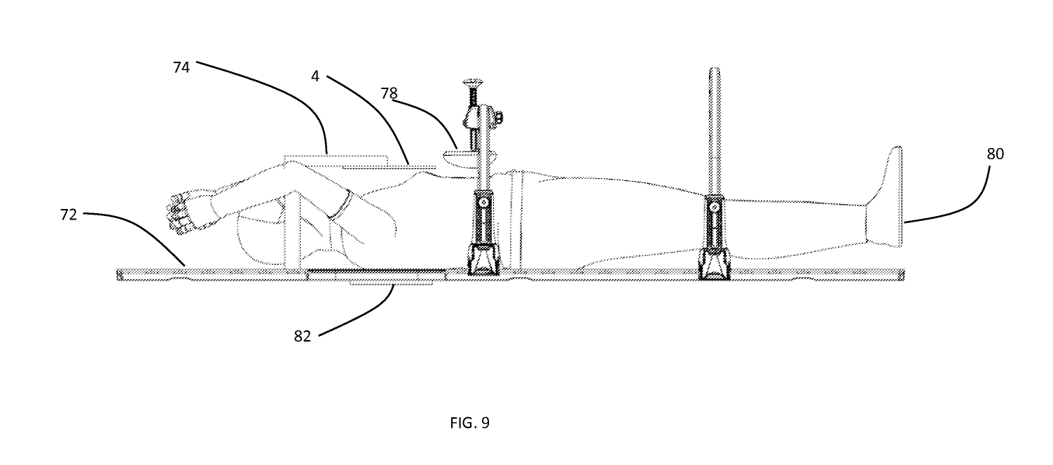

FIG. 9 shows a side view of patient 80 on a patient support platform 72. In this view the posterior coil 82 can be seen attached to the bottom of the patient support platform. This posterior coil is removably attached and can be positioned in multiple locations depending on tumor location and patient anatomy.

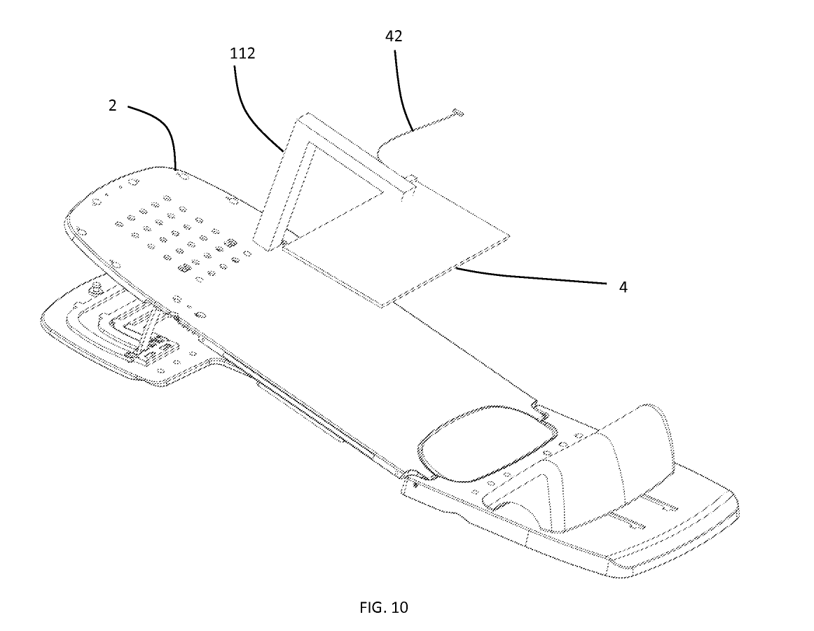

There are a number of ways the coil support can be configured. FIG. 10 shows one configuration in which the support 112 does not contain any adjustable pieces. The support can be mounted on the left or right of the patient platform 2. It can also have multiple attachment points to the platform such that the coil 4 can be adjusted superior or inferior with respect to the patient.

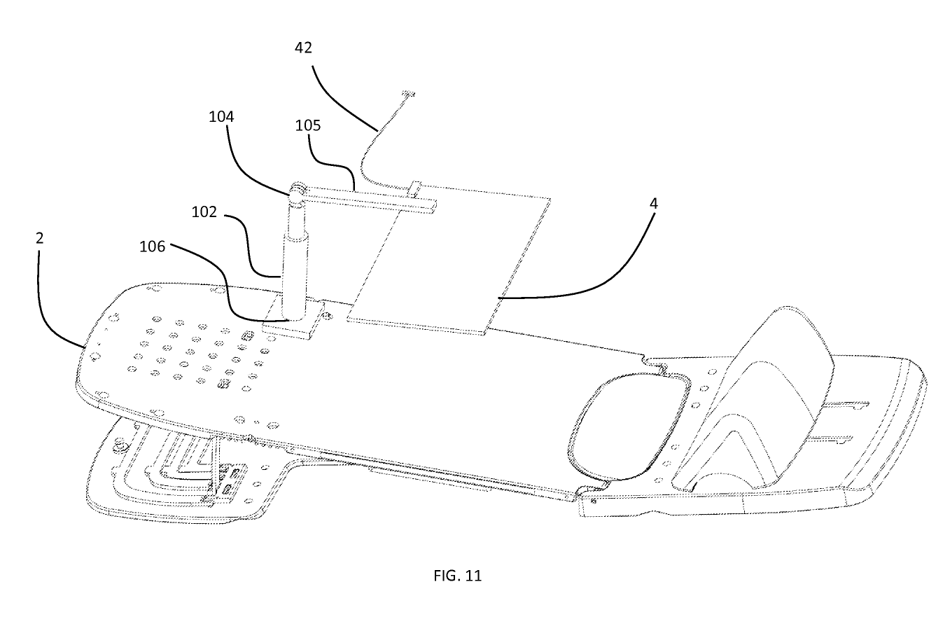

Alternatively, adjustment can be provided within the support. FIG. 11 illustrates an adjustable support. The platform mount 106 can be located at multiple locations on the platform 2. It can be mounted in such a way that it can be quickly connected and disconnected. The first member 102 is telescoping, allowing the coil 4 to be adjusted anteriorly and posteriorly. The telescoping configuration is preferred as this allow the overall height of the support to be kept to a minimum, allowing the coil to pass through the bore of the MRI without collision. The joint 104 is adjustable and can be locked into multiple locations allowing the angle of the coil to be adjusted. The second member 105 can also be configured to be telescoping to allow the coil to be adjusted superior or inferior with respect to the patient.

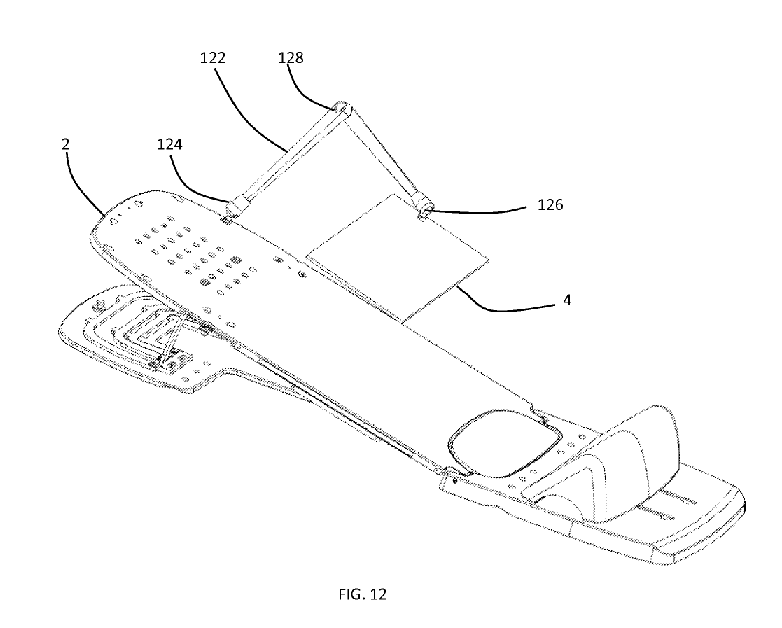

As shown in FIG. 12 the coil support 122 can also be an articulating arm. This allows for increased flexibility in positioning the coil. The arm can provide multiple degrees of freedom at the first knuckle 124 and the second knuckle 126. It can also rotate about the joint 128.

An important feature of any of these configurations is that the overall width of the patient and support structure is not increased. This allows the most versatility in fitting the device and patient through the bore of an MRI machine.

If the device is configured such that the patient is in the prone position the coil support positions the posterior coil and the inferior coil is attached to the bottom of the patient support platform.

The present invention is further defined by the following claims.

We claim:

1. A device for supporting and imaging a patient in an MRI machine in preparation for radiation therapy treatment comprising;
a. a platform with a top and bottom surface for supporting the patient;
b. at least one removable anterior coil set support mechanism attached to the platform; and
c. at least one anterior MRI coil attached to the coil set support mechanism and suspended above or below the patient;
wherein the MRI coil does not contact the patient, and wherein the MRI coil support attaches to the platform around the patient head or neck on at least one side of the patient head or neck.

2. The device of claim 1 wherein the at least one anterior coil is a rigid coil.

3. The device of claim 1 wherein the at least one anterior coil is a flexible coil.

4. The device of claim 1 further comprising a second MRI coil attached to the top or bottom surface of the platform, wherein the second coil is attached posterior to the patient.

5. The device of claim 4 wherein the second posterior coil is a rigid coil.

6. The device of claim 4 wherein the second posterior coil is a flexible coil.

7. The device of claim 1 wherein the platform is an angling back support.

8. The device of claim 1 compatible with diagnostic imaging, MRI, PET, CT, x-ray and radiation therapy treatment.

9. The device of claim 1 wherein the MRI coil is supported by at least one of; a rigid attachment, a telescoping cylinder, a rotatable member, or an articulating member.

10. The device of claim 4 wherein the MRI coil is supported by at least one of; a rigid attachment, a telescoping cylinder, a rotatable member, or an articulating member.

11. The device of claim 1 wherein the coil set support can be quickly and easily attached and removed from the platform.
